(19) United States
(12) Patent Application Publication
(10) Pub. No.: US 2010/0239722 A1
(43) Pub. Date: Sep. 23, 2010

(54) APPARATUS AND METHOD OF REDUCING CARRY OVER IN FOOD PROCESSING SYSTEMS AND METHODS

(76) Inventors: Sean A. Pendergast, Berlin, MA (US); Steven Moysey, South Grafton, MA (US); Patrick Mon, Milford, MA (US)

Correspondence Address: CESARI AND MCKENNA, LLP, 88 BLACK FALCON AVENUE, BOSTON, MA, 02210, US

(21) Appl. No.: 12/712,487

(22) Filed: Feb. 25, 2010

Related U.S. Application Data
(60) Provisional application No. 61/155,381, filed on Feb. 25, 2009.

(51) Int. Cl. G01N 33/02; A01J 11/00; A23G 9/22

(52) U.S. Cl. 426/231; 99/455; 222/146.6

(57) ABSTRACT

The present invention relates generally to a system and method of removing or at least minimizing carry over along food contact surfaces of a food processing and dispensing system. Specifically, data including color information related a first food product may be received and stored on a CPU. When the presence of carry over on the food contact surface is detected the CPU utilizes color information related to a second food product to determine whether to implement a purge cycle to remove the carry over along the food contact surface before the production of the second food products based on a calculated color variance between received color information related the first food product and received color information related to the second food product. If the calculated color variance is outside a predetermined range of values, the system implements a purge cycle before producing the second food product on the food contact surface.

### CROSS-REFERENCE TO RELATED APPLICATIONS

The present application claims the benefit of U.S. Provisional Patent Application Ser. No. 61/155,381, which was filed on Feb. 25, 2009, by Sean A. Pendergast for an APPARATUS AND METHOD OF REDUCING CARRY OVER IN FOOD PROCESSING SYSTEMS AND METHODS and is hereby incorporated by reference.

### BACKGROUND OF THE INVENTION

1. Field of the Invention

The invention relates generally to reducing carry over in food processing systems and methods and, more particularly, to a system and methods of removing or at least minimizing carry over along food contact surfaces of a food processing and dispensing system.

2. Background Information

Carry over in the food processing industry refers generally to the presence and/or the effects of residual food ingredients, components, intermediates and/or end products that remain after processing along food contact surfaces of food processing systems. Sometimes, carry over can affect the food products or intermediates that these systems produce. For instance, with respect to producing ice cream or frozen yogurt, residual ingredients, components, intermediates, and/or products themselves, which remain along food contact surfaces, may affect subsequent productions of ice cream or frozen yogurt with respect to certain qualities such as, for instance, taste, color, and texture. In a traditional ice cream shop context, carry over would include the presence of residual ice cream from one serving to the next on, for instance, utensils used to portion or scoop ice cream. Carry over in this context may be handled relatively easily. However, residual ice cream end products or ice cream ingredients, components and/or intermediates that are present along food contact surfaces of, for instance, an automated ice cream processing and dispensing system may be more difficult to handle and could have effects on the properties and qualities of ice cream produced, e.g., from one batch or serving to another. Removal of carry over, or at least minimizing carry over, along the food contact surfaces of such systems is therefore desirable.

For example, the inventions disclosed in the assignee's copending and related applications, including U.S. Patent Publication Nos: 2006/0054614; 2006/0162348; 2006/0162347; 2006/0003065; 2007/10251260 and PCT Application Nos.: WO 92/02146; WO 03/041513; WO 04/019707; and WO 06/076733, and the assignee's patents, including U.S. Pat. Nos. 5,292,030; 5,433,967; 5,473,909; 5,603,257; 5,727,713; 5,758,571; 5,868,065; 6,698,228; 6,745,595; 6,907,741; 6,941,858; 6,952,928; 7,052,728; and 7,131,279 which are hereby incorporated by reference also suffer from the carry over issues and thus may cause the flavor of the ice cream dispensed from the machines to be affected.

### SUMMARY OF THE INVENTION

Applicant's present invention overcomes the disadvantages of associated with carry over by providing an apparatus and method for removing or at least minimizing the presence and effects of carry over of food ingredients, components, intermediates and/or end products from one production cycle to another in food processing and dispensing system for producing chilled or at least partially frozen food products. More particularly, the system and methods of the invention may be incorporated or integrated with systems and methods for producing ice cream, frozen yogurt, and/or slushies.

In one embodiment of the illustrative invention, data including color information related a first food product to be produced by the food processing and dispensing system may be received and stored on a CPU. After the first food product has been produced and dispensed from the food processing and dispensing system, a sensor illustratively activated to detect the presence of carry over on the food contact surface. If carry over is detected by the sensor, the CPU may utilize stored color information related to a second food product to be produced by the processing and dispensing system to determine whether to implement a purge cycle to remove the carry over along the food contact surface before the production of the second food products. This determination is illustratively based on a calculated color variance between received color information related the first food product and color information related to the second food product. If the calculated color variance is outside a predetermined range of values, the system implements a carry over removal device, e.g. a scrapper to purge cycle before producing the second food product on the food contact surface. If, however the color range is within a predetermined range of values, the second food product is produced without implementing a purge cycle on the food contact surface.

### BRIEF DESCRIPTION OF THE DRAWINGS

The invention description below refers to the accompanying drawings, of which:

FIG. 1 is a schematic diagram of one aspect of the invention including a method of removing or at least reducing/minimizing carry over in a system and method of producing chilled or at least partially frozen food products;

FIG. 2 is a flow chart of another aspect of the invention which includes an operative method of removing or at least reducing/minimizing carry over as described with reference to FIG. 1;

FIG. 3 is a flow chart of an alternative embodiment of the present invention described with reference to FIG. 2;

### DETAILED DESCRIPTION OF AN ILLUSTRATIVE EMBODIMENT

Applicant's present invention overcomes the disadvantages of associated with carry over by providing an apparatus and method for removing or at least minimizing the presence and effects of carry over of food ingredients, components, intermediates and/or end products from one production cycle to another in food processing and dispensing system for producing chilled or at least partially frozen food products. More particularly, the system and methods calculate a color variance between a first food product and a second food product to determine whether to remove carry over that has been detected on the food contact surface. The illustrative embodiments of the present invention may be incorporated or integrated into systems and/or methods for producing ice cream, frozen yogurt, and/or slushies.

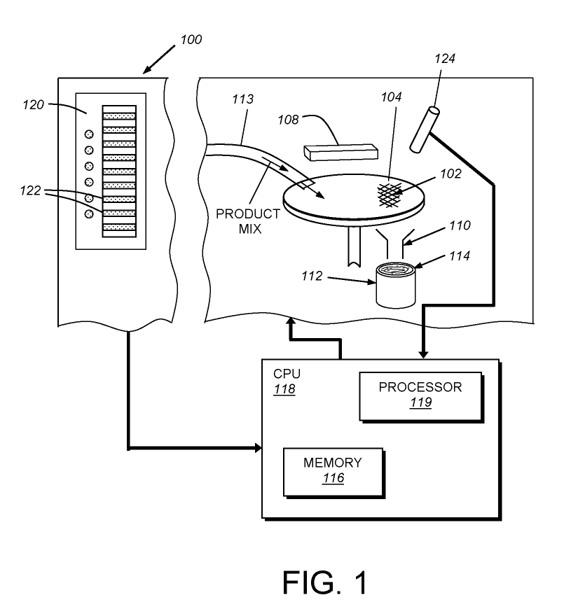

FIG. 1 is an illustrative embodiment of the present invention which illustrates an apparatus and/or system for removing or at least minimizing carry over of food product ingredients, components, intermediates, and/or end products from one production cycle to another in a processing and dispensing chilled or at least partially frozen food products, such as ice cream, frozen yogurt, and slushes. The illustrative embodiment may be used with any of the systems and methods disclosed in any of the above-incorporated patents and/or patent applications. The invention however is not limited to these systems and methods and envisions that the invention may be employed in other food processing systems and methods. In addition, the invention is not limited to producing chilled or at least partially frozen food products and may be employed in producing other food products and/or other food product ingredients, compositions, or intermediates. For purposes of disclosing the invention, the illustrative embodiment is described with reference to an apparatus, system, and method for producing ice cream, frozen yogurt, and/or slushies from one or more ingredients, components, and/or intermediates that are referred to collectively as a product mix. Product mix may include, for instance, a base mix for ice cream, yogurt, or slush, at least one flavoring and, optionally, one or more additives or mix-ins including, but not limited to, sundries, candies, etc. Carry over refers to at least residual product mix that may be included in end products, and/or that remains along the food contact surfaces of a processing and dispensing system. Food contact surfaces include, but are not limited to, delivery and mixing surfaces, chilling and freezing surfaces and/or dispensing surfaces.

The illustrative embodiment may employ a software program, a predetermined algorithm and/or associated hardware and/or firmware that detect the presence of carry over along one or more food contact surfaces of a processing and dispensing system, and that tracks the color of each processed product that the processing system produces. Depending on stored color information relating to an end food product the system produces during a first or previous production cycle and the color of a newly selected or second end product that the system will produce during at least a second or next production cycle, the apparatus or method prompts the system to implement a purge cycle between production cycles to remove or at least minimize carry over along food contact surfaces. In this manner, the processing system may perform multiple consecutive production cycles with minimal or no carry over from one cycle to another. The present invention helps to ensure that undesirable carry over is not present in, for instance, one or more consecutively produced batches or servings of ice cream, frozen yogurt and slush, and thereby helps to minimize the effects of carry over that may affect color, taste, texture, or other qualities of such products.

As shown in FIG. 1, in one embodiment of the invention, a sensor 124, e.g. an optical sensor, detects the presence of carry over 102 along a food contact surface 102, such as the processing or freezing surface or table 104 of a processing and dispensing system 100. The system 100 may include any of the systems described in the assignee's U.S. Patent Publication Nos: 2006/10054614; 2006/10162348; 2006/10162347; 2006/10003065; 2007/0251260 and PCT Application Nos.: WO 92/02146; WO 03/041513; WO 04/019707; and WO 061076733, and the assignee's patents, including U.S. Pat. Nos. 5,292,030; 5,433,967; 5,473,909; 5,603,257; 5,727,713; 5,758,571; 5,868,065; 6,698,228; 6,745,595; 6,907,741; 6,941,858; 6,952,928; 7,052,728; and 7,131,279.

Carry over detection may illustratively be implemented after a production cycle zo during which the system 100 processes a product mix 106 into ice cream, frozen yogurt and/or slush along the table 104. The product mix is dispensed onto the food contact surface via product introduction means 113. In this process, the system 100 removes the processed product mix 106 from the table 104 using a product removal device 108 which may, for example, scrape the product mix 106 from the table 104 and dispenses it through a dispensing means 110 into a container 112 as a batch or portioned serving 114. The product removal device 108 may illustratively be a scrapper or squeegee which removes the carry over off the food contact surface and into a dispensing means 110.

The illustrative processing and dispensing system tracks, e.g., records, and stores, information related to specifically correlated colors of batches or servings of ice cream, frozen yogurt and slush, and, optionally, information related to colors of residual carry over 102, e.g. along the table 104. The method uses the tracked information to determine whether the system 100 is to implement a purge cycle between production cycles to help to remove or at least minimize carry over 102, e.g., along the table 104.

In one embodiment of invention, the method uses color information related to at least one batch or serving of ice cream, frozen yogurt or slush that the system 100 has previously produced during a first or previous production cycle in relation to color information related to at least one subsequently selected batch or serving that the system 100 is to produce during at least a second or subsequent production cycle. The method uses received color information related to the first or previous product cycle and the second or subsequent production cycle to determine whether the system 100 is to implement a purge cycle between one or more production cycles. The purge cycle is implemented by a carry over removal device 115. In one embodiment of the present invention, color information that the method tracks and uses correlates to user selected flavorings, e.g., vanilla, chocolate, strawberry, coffee, mocha, raspberry, etc., that the system 100 is employed to produce.

As an illustrative example, the method of the invention tracks the color information related to a serving of chocolate ice cream that the system 100 has already produced and a subsequently selected batch or serving of vanilla ice cream that the system 100 is to produce to determine if the system 100 is to implement a purge cycle between production cycles and before producing vanilla ice cream. In this manner, the method removes or at least minimizes carry over in the system 100 that may remain from producing chocolate ice cream because the variation in color between the two products does not fall within a predetermined range of values. Because the color variation is sufficiently different between chocolate and vanilla, the system removes or minimizes residual chocolate, coffee, or mocha carry over and its effects in subsequently produced vanilla ice cream by implementing a purge cycle between each production.

As stated above the illustrative embodiment of the present invention may be implementing using software, e.g., executed by processor 119 and stored in memory 116 or on a computer readable medium, of a CPU or controller 118 to track information related to the color and/or corresponding flavoring of ice products. In one embodiment of the invention, such color information includes a color delta of a flavoring of at least one currently selected batch or serving and a color delta of a flavoring used in a previously made batch or serving. In one embodiment of the invention, a predetermined algorithm, e.g., stored in memory 116, is employed to determine whether the color delta of currently selected flavorings are, for instance, comparatively large with respect to color delta of flavorings of previously made batches or servings. More specifically, the predetermined algorithm utilizes color delta information to determine whether a color variance between two or more consecutively selected batches or servings is acceptable, e.g., within a given variance range or maximum variance value. When the algorithm determines a color variance is not acceptable, the algorithm provides instructions to the system 100 to implement a purge cycle between two or more consecutive production cycles.

Using the illustrative example given above, if the system determines that the color variance of the color delta of chocolate flavoring in relation to a color delta of vanilla flavoring is not acceptable, e.g., exceeds a given variance range or maximum variance value, the present invention prompts the food processing and dispensing system 100 to implement a purge cycle before initiating a production cycle of vanilla ice cream.

The color delta information tracked via software may include data the CPU or controller 118 receives from signals that a user selection interface 120 transmits to the CPU or controller 118. The interface 120 may be configured to allow an end-user or customer to select/specify one or more products and to select/specify one or more flavorings (color deltas) 122, e.g., vanilla, chocolate, strawberry, coffee, mocha, etc. The CPU or controller 118 may employ such data related to at least two consecutively selected flavorings (color deltas) to track and analyze the selected flavorings (color deltas) and determine whether a color variance between consecutively selected flavorings is acceptable. If the color variance is not acceptable, as mentioned, the CPU or controller 118 in accordance with the present invention prompts the food processing and dispensing system 100 to implement a purge cycle between the at least two consecutive production cycles.

As described above, detecting the presence of carry over 102 along one or more food contact surfaces 102 the sensor 124 may transmit a signal to the CPU or controller 118 to indicate detection of the presence of carry over 102 along the one or more food contact surfaces 102, as an indicator(s) or cue(s) that the system is to analyze color deltas of at least two consecutively selected flavorings. Alternatively, the system may also use data received from the user interface 120 related to two consecutively selected flavorings to analyze flavoring color deltas to determine whether the system 100 is to implement a purge cycle irrespective of the presence of carry over 102 along one or more food contact surfaces. Thus, in this embodiment, the purge cycle may be run even if the there is no carry over detected by the sensor 124 on the food contact surface.

In yet another embodiment of the invention, the method may use data received from the interface 120 related to two or more consecutively selected flavorings (color deltas), and, optionally, two or more consecutively selected base mixes, to determine whether the system 100 is to implement a purge cycle between production cycles. As an illustrative example of this embodiment, the method may determine the system 100 does not need to implement a purge cycle between a production cycle of chocolate ice cream and a production cycle of vanilla ice cream, but may determine the system 100 is to implement a purge cycle after two or more consecutive production cycles of chocolate ice cream, or chocolate and coffee ice cream, or coffee and mocha ice cream, etc. before initiating a production cycle of vanilla ice cream. In this case, carry over from one production cycle of chocolate, coffee or mocha ice cream may not affect a subsequent production cycle of vanilla ice cream, while two, or more consecutive production cycles of chocolate, coffee, and/or mocha ice cream may produce cumulative carry over that would affect a subsequent cycle of vanilla ice cream.

In one embodiment of the present invention, the purge cycle includes all of the stages, phases, or steps of a typical production cycle that the system 100 performs to produce a batch or serving of ice cream, yogurt, or slush. In accordance with the invention, during the purge cycle, the system 100 processes a predetermined volume of product mix that the system 100 will use to produce a subsequently selected batch or serving. The product mix or one or more materials suitable for removing the effects of carry over along one or more food contact is dispensed onto the food contact surface via product introduction means 113. Referring to the illustrative example described above, the method prompts the system 100 to process a predetermined volume of product mix that the system 100 will use to produce vanilla ice cream. The predetermined volume of product mix at least partially coats one or more food contact surfaces, e.g., the table 104, to help to remove or at least reduce/minimize carry over along such surfaces.

In another embodiment of the invention, the purge cycle may include only certain stages, phases, or steps of a production cycle that the system 100 performs to produce a product, while excluding other stages, phases, or steps, e.g., aeration of product mix or addition of mix-ins.

The predetermined volume of product mix that the system 100 processes during the purge cycle may include a volume of product mix that is sufficient to, as mentioned, at least partially coat one or more target food contact surfaces. In one embodiment of the invention, the predetermined volume of dispensed product mix is relatively small, e.g., about 2 ounces, in comparison to a volume of product mix that the system 100 will process to produce a batch or serving during a subsequent production cycle.

Alternatively, or additionally, in another embodiment of the invention a predetermined volume of one or more materials that are suitable for helping to remove or at least reduce/minimize carry over along one or more food contact surfaces including, but not limited to, propylene glycol, e.g., food grade propylene glycol. Alternatively, or additionally, in another embodiment of the invention, the method prompts the system 100 to process during the purge cycle a predetermined volume of product mix of a subsequently selected product, propylene glycol, other suitable materials, or combinations thereof.

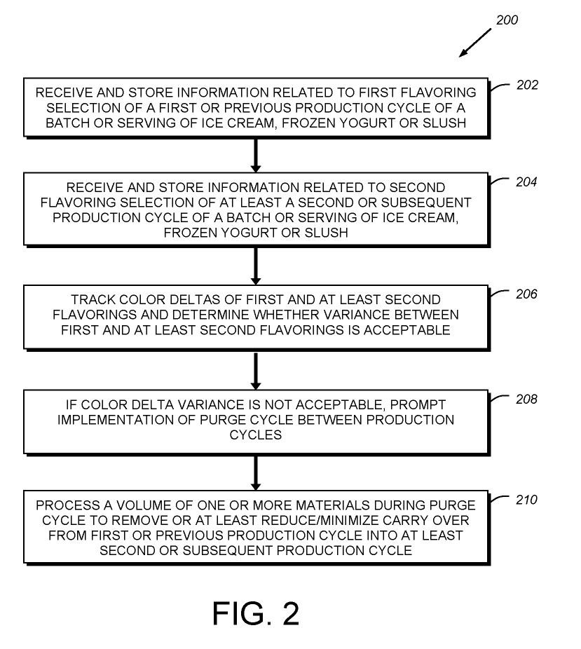

FIG. 2 is a flow chart illustrates an operating method 200 to remove or at least minimize carry over between production cycles, as described above. The method 200, however, is exemplary only and not limiting and thus may be altered, e.g., by having stages added, removed or rearranged.

At step 202, the CPU or controller 118 receives and stores data signals from the user interface 120 of the system 100 including at least information related to a first flavoring selection that an end user or customer selected via the interface 120 for a batch or serving of ice cream, frozen yogurt or slush the system 100 produced during a first or previous production cycle.

At step 204, the CPU or controller 118 receives and stores data signals from the interface 120 including at least information related to at least a second flavoring selection that an end user or customer selected via the interface 120 subsequent to the first flavoring selection for a subsequent batch or serving of ice cream, frozen yogurt or slush the system 100 is to produce during at least a second or subsequent production cycle.

At step 206, the CPU or controller 118 tracks color deltas of the first and at least second flavoring selections. Illustratively the predetermined algorithm stored in the CPU or controller 118 memory 116 determines whether the variance of the color deltas of the first and at least second flavoring selections are within a predetermined range of variances and/or maximum variance values.

At step 208, in the event the algorithm determines the color delta variance of the first and at least second flavoring selections is not acceptable, e.g., is not within a predetermined range of values, the CPU or controller 118 initiates instructions to prompt or cause the system 100 to implement a purge cycle between the first and the second production cycles, e.g., between two or more consecutive production cycles.

At step 210, during the purge cycle, the system 100 processes a volume of one or more materials, e.g., the second flavoring selection, propylene glycol, one or more other materials suitable for removing the effects of carry over along one or more food contact surfaces, and combinations thereof. In this manner, the system 100 at least partially coats at least some of the food contact surfaces to remove or at least minimize carry over along such food contact surfaces.

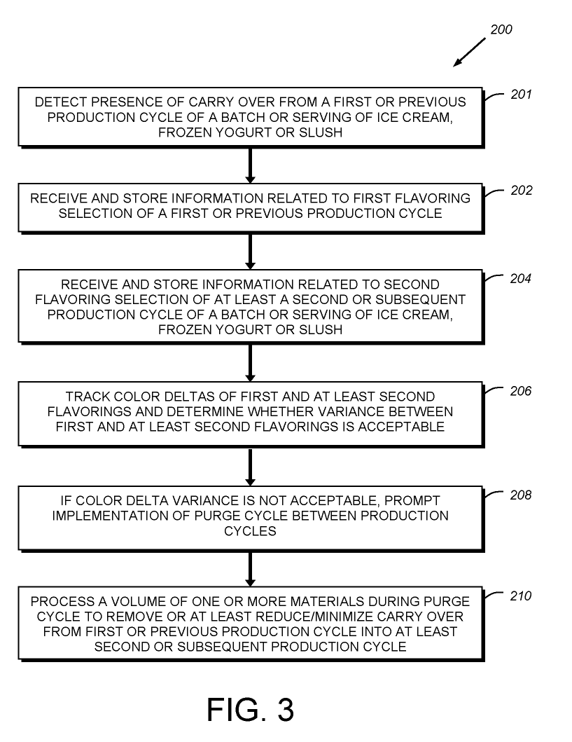

Referring to FIG. 3 and with further reference to FIGS. 1 and 2, the method 200 may also include one or more of steps 202 thru 210 described above with an additional step of 201. At stage 201, the system 100 detects, e.g., via the sensor 124, the presence of carry over along one or more food contact surfaces, e.g., a cold table, and the CPU or controller 118 receives detection signals the sensor 124 transmits that indicate the presence of carry over. The CPU or controller 118 is thereby signaled or cued to enact one or more of stages 202 thru 210 of the method 200 according to the invention to determine whether the system 100 is to implement a purge cycle between production cycles and to prompt the system 100 to implement the purge cycle as described above.

In describing aspects of the invention, specific terminology is used for the sake of clarity. For purposes of description, each specific term is intended to at least include all technical and functional equivalents that operate in a similar manner to accomplish a similar purpose. In some instances, where a particular aspect of the invention includes a plurality of system elements or method steps, those elements or steps may be replaced with a single element or step; likewise, a single element or step may be replaced with a plurality of elements or steps that serve the same purpose. Further, where parameters for various properties are specified herein for aspects of the inventions, those parameters can be adjusted or rounded-off to approximations thereof within the scope of the invention, unless otherwise specified.

Lastly the although the foregoing description has been directed to specific embodiments of this invention, it will be apparent, however, that other variations and modifications may be made to the described embodiments, with the attainment of some or all of their advantages. For instance, it is expressly contemplated that the teachings of this invention can be implemented as software, including a computer-readable medium having program instructions executing on a computer, hardware, firmware, or a combination thereof. Accordingly, this description is to be taken only by way of example and not to otherwise the scope of the invention. Therefore, it is the object of the appended claims to cover all such variations and modifications as come within the true spirit and scope of the invention.

What is claimed is:

1. A method comprising:
producing a first food product on a food contact surface of a processing and dispensing system;
receiving, at a processor, data including color information related to the first food product;
storing the data in a memory;
receiving, at the processor, data including color information related to a second food product to be produced by the processing and dispensing system;
determining whether to implement a purge cycle to remove food product carry over from the first food product before the production of the second food product based on a calculated color variance between received color information related to the first and second, wherein the calculated color variance is required to be outside a predetermined range of values in order to implement the purge cycle; and
in response to determining that the calculated color variance is outside the predetermined range of values, implementing a purge cycle before producing the second food product on the food contact surface.

2. The method of claim 1 comprising:
in response to determining that the calculated color variance is within the predetermined range of values, producing the second food product without implementing a purge cycle on the food contact surface.

3. The method of claim 1 further comprising:
detecting, by a sensor, a presence of carry over along the food contact surface of the processing and dispensing system.

4. The method of claim 1, wherein the color information correlates directly to the flavoring of a food product produced by the processing and dispensing system.

5. The method of claim 1, wherein the first and second food products are chilled food products which are mixed and chilled on the food contact surface according to a user selection on a user interface.

6. The method of claim 5 further comprising
transmitting a signal to the processor on a local computer from the user interface operatively connected to the processing and dispensing system, user interface configured to allow a customer to select a combination of a base product, a flavoring, and one or more mix-ins to be produced on the food contact surface.

7. The method of claim 1 wherein the purge cycle processes a predetermined volume of product mix to produce vanilla ice cream to at least partially coat the food contact surface to help minimize carry over along the food contact surface.

8. The method of claim 1, wherein the food contact surface is a cold table used for making ice cream.

9. A computer readable medium containing executable program instructions executed by a processor, comprising:
program instructions that produce a first food product on a food contact surface of a processing and dispensing system;
program instructions that data including color information related to the first food product;
program instructions the data in a memory;
program instructions that receive, at the processor, data including color information related to a second food product to be produced by the processing and dispensing system;
program instructions that determine whether to implement a purge cycle to remove food product carry over from the first food product before the production of the second food product based on a calculated color variance between received color information related to the first and second, wherein the calculated color variance is required to be outside a predetermined range of values in order to implement the purge cycle; and
program instructions that implement a purge cycle before producing the second food product on the food contact surface in response to a determination that the calculated color variance is outside the predetermined range of values.

10. The computer readable medium of claim 9 further comprising:
program instructions that produce the second food product without implementing a purge cycle on the food contact surface in response to a determination that the calculated color variance is within the predetermined range of values.

11. The computer readable medium of claim 10, the program instructions further comprising:
program instructions that detect a presence of carry over along the food contact surface.

12. The computer readable medium of claim 11 further comprising
program instructions that transmit a signal to the processor on a local computer from the user interface operatively connected to the processing and dispensing system, user interface configured to allow a customer to select a combination of a base product, a flavoring, and one or more mix-ins to be produced on the food contact surface.

13. An apparatus comprising:
a food contact surface configured to produce at least a first food product and a second food product; and
a CPU configured to
(i) receive data, the data including color information related the first food product,
(ii) store the data,
(iii) receive data including color information related to a second food product to be produced by the processing and dispensing system,
(iv) determine whether to implement a purge cycle to remove food product carry over from the first food product before the production of the second food product based on a calculated color variance between received color information related to the first and second, wherein the calculated color variance is required to be outside a predetermined range of values in order to implement the purge cycle, and
(v) implement a purge cycle to remove carry over from the food contact surface in response to a determination that the calculated color variance is outside the predetermined range of values.

14. The apparatus of claim 13 wherein the food contact surface is further configured to:
produce the second food product without implementing a purge cycle on the food contact surface in response to a determination that the calculated color variance is within the predetermined range of values.

15. The apparatus of claim 13 wherein a sensor is configured to detect a presence of carry over along the food contact surface.

16. The apparatus of claim 13, wherein the color information correlates directly to the flavoring of a food product produced by the processing and dispensing system.

17. The apparatus of claim 13, wherein the first and second food products are chilled food products which are mixed and chilled on the food contact surface according to a user selection on a user interface.

18. The apparatus of claim 17 further comprising a user interface configured to transmit a signal to a processor on the CPU, user interface configured to allow a customer to select a combination of a base product, a flavoring, and one or more mix-ins to be produced on the food contact surface.

19. The apparatus of claim 13 wherein the apparatus is a processing and dispensing system for user customizable chilled food products.

20. The apparatus of claim 13, wherein the purge cycle processes a predetermined volume of product mix to produce vanilla ice cream to at least partially coat the food contact surface to help minimize carry over along the food contact surface.
